(19) United States
(12) Patent Application Publication
(10) Pub. No.: US 2019/0151495 A1
(43) Pub. Date: May 23, 2019

(54) Composite biomaterials with controlled release of active ingredient, preparation process and uses

(71) Applicants: CENTRE NATIONAL DE LA RECHERCHE SCIENTIFIQUE, PARIS (FR); SORBONNE UNIVERSITE, PARIS 6 (FR)

(72) Inventors: Christophe HELARY, CHAMPIGNY SUR MARNE (FR); Thibaud CORADIN, PARIS (FR)

(21) Appl. No.: 16/350,064

(22) PCT Filed: Feb. 20, 2017
(86) PCT No.: PCT/FR2017/050377

§ 371 Date: Aug. 20, 2018

(30) Foreign Application Priority Data
Feb. 22, 2016 (FR) 1651432

(51) Int. Cl. A61L 15/32; A61L 15/26; A61L 15/44; A61L 15/60; A61L 31/12; A61L 31/14; A61L 31/16

(52) U.S. Cl. CPC A61L 15/325; A61L 15/26; A61L 15/44; A61L 15/60; A61L 2300/412; A61L 31/145; A61L 31/16; A61L 2300/41; A61L 2300/406; A61L 31/129

(57) ABSTRACT

The invention relates to a composite biomaterial based on collagen, on at least one hydrophobic organic polymer and on at least one active ingredient, the process for preparing same, a dressing comprising such a composite biomaterial, an abdominal wall reinforcement comprising such a composite biomaterial, and also the uses of said composite biomateral, especially in the therapeutic field.

The invention relates to a composite biomaterial based on collagen, on at least one hydrophobic organic polymer and on at least one active ingredient, the process for preparing same, a dressing comprising such a composite biomaterial, an abdominal wall reinforcement comprising such a composite biomaterial, and also the uses of said composite biomaterial, especially in the therapeutic field.

The invention applies in particular to the field of biomaterials which make it possible to deliver active ingredients, especially to care for chronic injuries.

The skin, which covers the whole of the human body, performs the role of a barrier protecting organs and tissues from external attacks. Its role is therefore essential for the correct functioning of the organism. The skin is composed of three layers: the epidermis, the dermis and the hypodermis or subcutaneous layer. After an injury, tissue repair or wound healing begins immediately. It is composed of four phases: haemostasis, the inflammatory phase, the proliferative phase and the remodelling phase. A chronic injury is defined as a skin wound which has not reclosed after 42 days. These wounds may take the form of leg ulcers, foot ulcers in diabetic patients, or pressure sores. These chronic wounds result from complications in patients suffering from diabetes, vascular insufficiency or else nutritional deficiencies. The prevalence of foot lesions in diabetics is approximately 20% each year. Every year, this problem affects several million people around the world, commonly aged over 65, and therefore constitutes a major issue for research in regenerative medicine. The main difference with what are referred to as acute injuries is that the wound healing process is not complete and remains stuck in the inflammatory phase. Treatments for chronic injuries must both stop the inflammation and promote tissue regeneration in order to obtain closure of the wound and also the healing thereof.

The technique commonly used to care for chronic wounds is lancing of the wound followed by compression thereof. This involves removing the necrotic tissue and the fibrous adhesions that have developed between the tissues in order to be able to clean the wound. In some cases, this technique is ineffective and it is then necessary to cover the wound with a dressing based on biomaterials such as hydrocolloids or hydrogels. The function of the dressing is to protect the wound from potential infections while enabling closure thereof. Hydrocolloids are defined in the assessment report by the Haute Autorité de Santé (French High Authority of Health) as dressings consisting of absorbent polymers, the physico-chemical properties of which are associated with the presence of carboxymethyl cellulose. However, this type of dressing may give rise to a risk of superinfection associated with the excessively moist micro-environment that it generates. Hydrogels are physical or chemical gels composed of polymer chains swollen by a large amount of water (approximately 70% of the total volume of the hydrogel). It is known to use hydrogels based on alginate or collagen. However, alginate breaks down quite quickly on contact with the enzymes of the human body. Collagen has limited intrinsic mechanical properties which may be improved by crosslinking using chemical agents (aldehydes, carbodiimides, etc.). However, the toxicity of these chemical agents is problematic, since they may give rise to undesirable side effects.

The dressings may be used in combination with an oral treatment or a topical composition based on at least one active ingredient of antibiotic or anti-Inflammatory type, or a molecule promoting wound healing. However, the oral treatment may prove ineffective due to the limited access of the active ingredient to the epidermis; and the topical composition generally requires frequent cutaneous applications and direct contact between the injury and the air, increasing the risk of infection.

Thus, the solutions proposed above are either ineffective and the wound treatment then requires the use of a skin substitute which is very costly, or they do not act on all the steps of wound healing and require the combination of several of these solutions.

Another solution is the prolonged release of an active ingredient using a suitable support for enabling absorption, in a manner that is regular and sustained over time, of said active ingredient. The release needs to be controlled in order for the medicament to be as effective as possible, with as few side effects as possible and a prolonged duration of action. Moreover, pure collagen hydrogels have been described as support for the controlled release of an active ingredient. However, pure collagen hydrogels do not sufficiently retain the active ingredient, and they have insufficient mechanical strength [Wallace et al., Advanced Drug Delivery Reviews, 2003, 55, 1631-1649].

Moreover, collagen-organic polymer composite materials have been proposed for the prolonged release of medicaments. In particular, Ruszczak et al. [Advanced Drug Delivery Reviews, 2003, 55, 1679-1698] described a composite material based on a collagen sponge, on microparticles of poly(lactic-co-glycolic) acid (PLGA) and on an antibiotic as active ingredient. The composite material in the form of a collagen composite sponge was prepared as follows: microparticles of PLGA comprising the antibiotic were prepared by the double emulsion technique. The microparticles obtained were then mixed with an aqueous solution of collagen and of antibiotic, then the resulting dispersion was lyophilized. In Ruszczak et al., the insoluble collagen (IC) which serves as base constituent is dissolved in acetic acid at pH 4.5. At this pH, the initially fibrillar collagen dissolves in the acid in the form of triple helices of collagen, and loses its fibrillar form. Since the manufacturing process is based on lyophilization of the solution without passing back to a neutral pH, the final material is only formed of triple helices of collagen. Moreover, it is known that the collagen of collagen sponges does not have a fibrillar and striated structure, as reported for example by Vigier et al., Journal of Biomedical Materials Research, 2010, 94A, 2, 556-567. In addition, collagen sponges are known to be denatured in gelatin at 37° C. if they are not crosslinked by chemical agents. The result of this is that the composite material of Ruszczak et al. does not have properties of controlled and prolonged release of the active ingredient (without a rapid initial release) and optimized mechanical properties.

Other known collagen-organic polymer composite materials are in the form of fibres of said organic polymer covered with a thin layer of collagen.

However, the composite materials of the prior art do not have an optimized profile of release of the active ingredient (e.g. too quick and/or non-linear release). Moreover, they do not have suitable mechanical properties, especially suitable elasticity, elongation at break and/or breaking strength, to be able to be used in therapeutic dressings (e.g. extremely stiff materials or materials that are too slack and/or too fragile).

The aim of the present invention is to overcome the drawbacks of the prior art and to provide a biodegradable biocompatible material which has suitable mechanical properties to be able to be used in the field of therapeutic dressings, which is able to promote several steps of the wound healing process, especially in the case of chronic injuries, and which enables the controlled release of at least one active ingredient participating in said wound healing process or in tissue repair.

The subject of the invention is therefore a synthetic composite biomaterial comprising collagen, at least one organic polymer and at least one active ingredient, characterized in that:
the organic polymer is biodegradable, biocompatible, hydrophobic and has a glass transition temperature of less than or equal to 50° C., and preferably less than or equal to approximately 45° C., and a mean molar mass ranging from approximately 5 to 120 kDa,
the collagen is in the form of striated fibrils, in which the periodicity of the striations is 67 nm,
the collagen/organic polymer weight ratio ranges from approximately 10/1 to 1/3, preferably from approximately 5/1 to 1/2, and more preferably still from approximately 2/1 to 2/3,
the active ingredient is a hydrophobic active ingredient chosen from anti-inflammatories, antibiotics, compounds promoting tissue repair or wound healing, and a mixture thereof.

The synthetic composite biomaterial of the invention is biocompatible, biodegradable and has mechanical properties that are comparable to, or improved compared to, a pure collagen hydrogel, while enabling the controlled and prolonged release of one or more active ingredients, especially without a rapid initial release.

In particular, the composite biomaterial of the invention may virtually constantly release an active ingredient over a period of at least 7 days, and preferably over a period of at least 3 weeks or even 1 month.

The collagen of the composite biomaterial is in the form of striated fibrils and the collagen/organic polymer weight ratio ranges from approximately 10/1 to 1/3, preferably from approximately 5/1 to 1/2, and more preferably still from approximately 2/1 to 2/3. By virtue of these characteristics, the biomaterial is homogeneous and has good mechanical properties. In particular, the striated fibrillar structure is stable up to at least 50° C. and does not denature at the temperature of the human body (i.e. approximately 37° C.). Moreover, the biomaterial may release one or more active ingredients in a controlled, prolonged and virtually constant manner, especially while avoiding the phenomenon of rapid initial release of the active ingredient (also well-known as “burst release”).

The composite biomaterial of the invention is preferably devoid of any organic solvent.

The composite biomaterial of the invention may be in the form of a composite hydrogel or in the form of a dry composite material.

When the composite biomaterial is in the form of a dry composite material, it comprises at most approximately 10% by weight of water, and preferably at most approximately 5% by weight, relative to the total weight of the composite biomaterial. According to a preferred embodiment of the invention, the dry composite material does not comprise any solvation water.

When the composite biomaterial is in the form of a composite hydrogel, it comprises approximately from 70 to 95% by weight of water, and preferably 9% approximately from 80 to 90% by weight of water relative to the total weight of the composite biomaterial.

The composite biomaterial of the invention preferably consists of one or more active ingredients, one or more organic polymers, collagen and optionally water or an aqueous solution with a pH ranging approximately from 7 to 8 (e.g. phosphate-buffered saline).

In the present invention, the expression “biodegradable organic polymer” means an organic polymer which can be broken down or digested by microorganisms (e.g. bacteria, fungi, algae), especially by the action of enzymes. The reactions involved during biodegradation in humans are hydrolysis reactions, that is to say the breaking of covalent bonds by reaction with water (cf. current standard NFEN 13 432).

In the present invention, the expression “biocompatible organic polymer” means an organic polymer having the ability to not interfere with, and to not degrade, the biological environment in which they are used. In particular, they must not cause a strong inflammatory reaction (e.g. allergies) and/or must not be toxic to humans.

In the present invention, the expression “hydrophobic organic polymer” means an organic polymer for which a contact angle is measured approximately between 60° and 150°, preferably approximately between 60 and 110°, and more preferentially still approximately between 65 and 90°, for example according to the Wilhelmy measurement method detailed in the examples below.

Thus, the organic polymers in accordance with the invention are insoluble in biological fluids.

The presence of at least one hydrophobic organic polymer in the composite biomaterial of the invention enables the encapsulation of one or more active ingredients and the controlled release thereof.

The organic polymer preferably has a glass transition temperature of less than or equal to approximately 40° C., and more preferably still less than or equal to approximately 38° C.

Moreover, it is advantageous for the organic polymer to have a glass transition temperature, when it is combined with collagen, which is less than or equal to approximately 37° C., preferably less than or equal to approximately 32° C., and more preferably still less than or equal to approximately 28° C.

This is because a temperature of approximately 28° C. corresponds to the equilibrium temperature of a dressing on the skin (temperature at the ambient air-physiological temperature interface). A glass transition temperature of the organic polymer, when it is combined with collagen, of less than or equal to approximately 40° C. makes it possible to obtain a composite biomaterial having comparable, or even improved, mechanical properties compared to pure collagen (elasticity, elongation at break and/or breaking strength).

The organic polymer may be chosen from aliphatic polyesters, polyethylene glycols, polyanhydrides and poly(ortho-esters).

Preferably, the organic polymer is an aliphatic polyester, especially chosen from a polyglycolide [i.e. poly(glycolic acid) or PGA], a polylactide [i.e. poly(lactic acid) or PLA], a copolymer of glycolide and lactide (PLGA), a polylactone (e.g. poly(ε-caprolactone)), and a polyhydroxyalkanoate (e.g. polyhydroxyvalerate, poly(hydroxybutyrate)).

According to a particularly preferred embodiment of the invention, the organic polymer is a copolymer of glycolide and lactide.

The copolymer of glycolide and lactide may have a lactide/glycolide molar ratio ranging approximately from 50/50 to 85/15, preferably ranging approximately from 50/50 to 65/35.

The organic polymer may have a mean molar mass ranging approximately from 5 kDa to 60 kDa, and more preferably still approximately from 7 kDa to 20 kDa.

The content of organic polymer of the composite biomaterial, when it is in the form of a composite hydrogel, is at least approximately 10 mg/ml, preferably ranges approximately from 20 to 100 mg/ml, and more preferably still ranges approximately from 30 to 60 mg/ml.

The organic polymer of the composite biomaterial of the invention is in the form of nanodomains, especially having a mean size of less than or equal to approximately 700 nm, preferably less than or equal to approximately 600 nm, and more preferably still ranging approximately from 100 to 500 nm.

The mean size of these nanodomains is determined by transmission electron microscopy (TEM).

In other words, the organic polymer is not in the form of fibres or of microparticles, as is the case in the composite materials of the prior art.

The collagen is preferably a type I or III collagen, and more preferably still a type I collagen. Fibrillar type I collagen is a natural protein present at approximately 70% by weight in the extracellular matrix of the skin; it contributes to the mechanical structure of connective tissues and is the substrate for cell adhesion.

The content of collagen of the composite biomaterial, when it is in the form of a composite hydrogel, is at least approximately 10 mg/ml, preferably approximately from 20 to 100 mg/ml, and more preferably still approximately from 30 to 60 mg/ml.

A minimum collagen content of at least approximately 10 mg/ml in the composite biomaterial makes it possible to avoid the rapid degradation thereof in the physiological environment during cutaneous application (e.g. in the form of a dressing).

In the material of the invention, said fibrils are generally not ordered. The material therefore predominantly, or even solely, comprises isotropic domains.

The active ingredient of antibiotic type may be gentamicin, rifamycin or amoxicillin.

The active ingredient of anti-inflammatory type may be dexamethasone or an analgesic such as ibuprofen.

The active ingredient promoting wound healing or tissue repair may be spironolactone.

Spironolactone is known to be a mineralocorticoid receptor antagonist and to promote the repair of skin wounds.

According to a particularly preferred embodiment of the invention, the active ingredient is spironolactone.

In the present invention, the expression “hydrophobic active Ingredient” means an active ingredient having a partition coefficient P in an octanol/water system such that log P>0, preferably log P>2, and more preferably still log P>3.

The partition coefficient P is well known to those skilled in the art. It is equal to the ratio of the concentrations of a solute (active ingredient) in two phases: P=C′/C, C′ corresponding to the concentration of the solute in an organic solvent saturated with water and C corresponding to the concentration of the solute in water saturated with organic solvent.

The water-solubility of a hydrophobic active ingredient such as that used in the composite biomaterial of the invention is generally less than approximately 50 mg/l.

The content of active ingredient in the composite biomaterial depends on the active ingredient that it is wished to incorporate, and hence on the targeted application.

In particular, the composite biomaterial in the form of a composite hydrogel may comprise approximately from 0.1 to 10 mg/ml of spironolactone.

The composite biomaterial of the invention, in the form of a composite hydrogel, may have an elasticity (i.e. Young's modulus) of at least approximately 10 000 Pa, preferably at least approximately 40 000 Pa, and more preferably still ranging approximately from 50 000 to 100 000 Pa.

The composite biomaterial of the invention, in the form of a composite hydrogel, may have a shear modulus of at least approximately 3000 Pa, preferably at least approximately 5000 Pa, and more preferably still ranging approximately from 10 000 to 60 000 Pa, at a frequency of between 1 and 10 Hz.

The composite biomaterial of the invention may have an elongation at break of at least approximately 10%, preferably at least approximately 30%, and more preferably still ranging approximately from 40 to 75%.

The composite biomaterial of the invention may have a breaking strength of at least approximately 2.5 MPa, preferably at least approximately 3 MPa, and more preferably still ranging approximately from 3.5 to 12 MPa.

The composite biomaterial of the invention is in the form of a matrix of striated fibrils of collagen in which nanodomains formed of said organic polymer are homogeneously dispersed (homogeneous three-dimensional network). In particular, the nanodomains of said organic polymer have a mean size of less than or equal to approximately 700 nm, preferably less than or equal to approximately 600 nm, and more preferably still ranging approximately from 100 to 500 nm.

Another subject of the invention is a process for preparing a composite biomaterial as defined in the first subject of the invention, characterized in that it comprises at least the following steps:

i) preparing a hydrogel of collagen in the form of striated fibrils in which the periodicity of the striations is 67 nm, the concentration of collagen in the hydrogel being at least approximately 10 mg/ml,

ii) dehydrating the hydrogel as prepared in step i) by incubating said hydrogel in several successive mixed solutions [organic solvent/aqueous solvent] having an increasing proportion of organic solvent, said aqueous and organic solvents being miscible, followed by a final incubation in a pure solution of said organic solvent,

iii) bringing the dehydrated hydrogel from step ii) into contact with an impregnation solution comprising at least one organic polymer and at least one active ingredient as defined in the first subject of the invention, the volume of impregnation solution:volume of dehydrated hydrogel volume ratio being greater than or equal to approximately 3,

iv) rinsing the impregnated hydrogel from step ill) with an organic solvent, then with an aqueous solvent.

Thus, in the process of the invention, the fibrillar and striated structure of the collagen is preserved. The process is simple and economical and it makes it possible to obtain a biodegradable biocompatible material which has suitable mechanical properties to be able to be used in the field of therapeutic dressings, which is able to promote several steps of the wound healing process, especially in the case of chronic injuries, and which enables the controlled release of at least one active ingredient participating in said wound healing process or in tissue repair.

Preferably, the concentration of collagen in the hydrogel resulting from step i) ranges approximately from 20 to 100 mg/ml, and more preferably still approximately from 30 to 60 mg/ml. A concentration from approximately 30 to 60 mg/ml is particularly suitable for obtaining a composite biomaterial having a large capacity for hydration and swelling, good elasticity and good stability against enzymatic degradation.

Step i) may be carried out according to the following sub-steps:

i-1) preparing a solution of acid-soluble collagen, the collagen content of which varies approximately from 1 to 5 mg/ml, and is preferably of the order of 5 mg/ml,

i-2) evaporating the solution from step i-1) in air, and

i-3) bringing the solution from step i-2) into contact with a base.

Step i-1) may be carried out by any means known to those skilled in the art, especially by extraction of collagen from a bovine, murine or porcine species.

An acid-soluble solution is an acid aqueous solution in which the collagen may be dissolved. The pH thereof is generally less than approximately 4, preferably less than approximately 3, in the presence of acids, preferably acetic acid for example at approximately 0.5 M.

Step i-2) is gradual and enables the collagen within the solution to be concentrated. It may last approximately from 3 days to 2 weeks. At the end of sub-step 12), the concentration of the collagen solution is at least approximately 10 mg/ml, preferably approximately from 20 to 100 mg/ml, and more preferably still approximately from 30 to 60 mg/ml (i.e. the collagen has the desired concentration to form the composite biomaterial of the invention).

Step i-3) makes it possible to subject the concentrated collagen solution to an increase in pH which induces gelling of the collagen and the fibrillogenesis thereof.

It is carried out advantageously by bringing the solution from step i-2) into contact with a basic gaseous atmosphere, in particular an atmosphere of NH₃ or of (NH₄)₂CO₃.

Step i-3) generally lasts between approximately 12 h and 24 h.

Step i) may also comprise a sub-step i-4) of rinsing the hydrogel from step i-3), especially with several phosphate-buffered saline (PBS) solutions, in order to eliminate the base.

Step i) may also comprise a sub-step i-2′ between sub-steps i-2) and i-3), during which the solution from step i-2) is decanted into a mould and centrifuged. This step i-21 makes it possible to flatten out surface irregularities of the concentrated (and viscous) solution of collagen from step i-2).

Step i) may also be carried out according to the procedure as described in Wang et al., Nature Materials, 2012, 11, 724-733.

Step ii) makes it possible to lead to a dehydrated (i.e. water-free) material of pure collagen.

In a particular embodiment, the organic solvent is chosen from tetrahydrofuran (THF) and dimethylsulfoxide (DMSO).

In a particular embodiment, the aqueous solvent is chosen from water and a phosphate-buffered saline (PBS).

Step ii) may be carried out by incubating the hydrogel from step i) in a mixed organic solvent/aqueous solvent solution in which the content of organic solvent is approximately from 20 to 30% by volume, then in a mixed organic solvent/aqueous solvent solution in which the content of organic solvent is approximately from 40 to 50% by volume, then in a mixed organic solvent/aqueous solvent solution in which the content of organic solvent is approximately from 60 to 70% by volume, then in a mixed organic solvent/aqueous solvent solution in which the content of organic solvent is approximately from 80 to 95% by volume, then in a pure solution of said organic solvent.

The incubation in each of the solutions may last approximately from 30 min to 2 h, and preferably approximately from 45 min to 1 h 15, and more preferably still of the order of approximately 1 h.

Step ii) makes it possible to create and/or retain homogeneous porosity within the collagen. This step ii) is essential in the process of the invention since it makes it possible on the one hand to have reproducible results and on the other hand to obtain good impregnation of the hydrogel during the subsequent step iii). Thus, a homogeneous composite biomaterial is obtained (e.g. absence of microscopic domains of organic polymer in the composite biomaterial and retention of the striated fibrillar structure of the collagen) which has good properties of prolonged release of the active ingredient, while guaranteeing good mechanical properties.

Step iii) makes it possible to impregnate the hydrogel of step ii) with a solution of organic polymer and active ingredient.

By virtue of the porosity of the hydrogel and of a medium favourable to the diffusion of the organic polymer, a large amount of organic polymer may be incorporated into the hydrogel.

Step iii) is preferably carried out by preparing an impregnation solution comprising the active ingredient and the organic polymer, then incubating the dehydrated pure collagen hydrogel from step ii) in the impregnation solution.

In particular, the impregnation solution is prepared by dissolving the active ingredient at the desired concentration in an organic solvent then by adding, to the preceding solution, the organic polymer at the desired concentration, and finally by dissolving it in said preceding solution.

Step iii) is preferably carried out at room temperature.

The organic solvent may be chosen from all solvents which make it possible to dissolve both the active ingredient and the organic polymer. By way of example, the organic solvent may be THF or DMSO.

The concentration of organic polymer of the impregnation solution may range approximately from 20 to 500 mg/ml and preferably approximately from 80 to 160 mg/ml.

The concentration of active ingredient of the impregnation solution may vary approximately from 0.1 to 100 mg/ml, preferably approximately from 0.5 to 40 mg/ml, and more preferably still approximately from 1 to 10 mg/ml.

The volume of impregnation solution:volume of dehydrated hydrogel volume ratio preferably varies approximately from 3/1 to 20/1 and is preferably of the order of 5/1.

Step iii) generally lasts between approximately 12 h and 24 h.

The organic solvent of step iv) may be THF or DMSO.

The aqueous solvent of step iv) may be a phosphate-buffered saline (PBS), a fluid that simulates body fluids (well-known as serum body fluid or SBF), water or a cell culture medium.

Step iv) is preferably carried out by rinsing the impregnated hydrogel from step iii) in several baths of organic solvent, especially for approximately 15 seconds to 1 minute, and preferably for approximately 30 seconds; and by rinsing the impregnated hydrogel from step iii) in several baths of aqueous solvent, especially for approximately 15 minutes to 1 hour, and preferably for approximately 30 minutes.

Rinsing with an organic solvent makes it possible to eliminate excess polymer which has not been absorbed by the collagen hydrogel.

Rinsing with an aqueous solvent makes it possible to fix the polymer within the collagen hydrogel. Without this final step of rinsing with an aqueous solvent, the composite biomaterial does not have sufficient mechanical strength, and is fragile.

At the end of step iv), the composite biomaterial is in the form of a composite hydrogel.

The process may also comprise a step v) of lyophilization of the composite biomaterial from step iv).

At the end of step v), the composite biomaterial is in the form of a dry composite material. The dry composite material is preferably devoid of any organic or aqueous solvent.

The dry composite material may then be conserved for a long period.

The process may also comprise a step vi) of hydration of the dry composite material. This step vi) makes it possible to reform the composite material in the form of a composite hydrogel.

Step vi) may be carried out in the presence of an aqueous solution chosen from water, a phosphate-buffered saline, a fluid that simulates body fluids and a cell culture medium.

Steps v) and vi) make it possible to completely eliminate any organic solvent which could possibly have still been present in the composite hydrogel obtained at the end of the rinsing step iv).

The third subject of the invention is a composite biomaterial in accordance with the first subject of the invention, for medical use thereof.

The fourth subject of the invention is a composite biomaterial in accordance with the first subject of the invention, for use thereof in the treatment of chronic wounds. These chronic wounds are generally encountered in foot ulcers, venous ulcers or pressure sores.

In a particular embodiment, the composite biomaterial in accordance with the invention is left in contact with the skin for at least one week.

The fifth subject of the invention is a composite biomaterial in accordance with the first subject of the invention, for use thereof in the preventative treatment of infections after cardiac or colorectal surgery.

The sixth subject of the invention is a therapeutic dressing comprising an internal layer and an external layer consisting of a secondary dressing chosen from an adhesive, a compress, a bandage and a mixture thereof, characterized in that the internal layer comprises a composite biomaterial in accordance with the first subject of the invention.

The secondary dressing may in particular be a porous dressing, sterile gauze, a polyurethane foam or a film, or a polyamide/polyester nonwoven film.

The internal layer is in contact with the skin and the external layer maintains the internal layer.

The dressing may release the active ingredient, and in particular spironolactone, at a constant dose for at least one week.

The seventh subject of the invention is a composite biomaterial in accordance with the first subject of the invention, for use thereof in the treatment of hernias of the abdominal wall or eventrations.

The eighth subject of the invention is an abdominal wall reinforcement, comprising a composite biomaterial in accordance with the first subject of the invention.

### EXAMPLES

The starting materials used in the examples are listed below:
PLGA 7-17 KDa, 719897-5G, Sigma, 50:50 (Lactide:Glycolide),
PLGA 24-38 KDa, 719870-5G, Sigma, 50:50 (Lactide:Glycolide),
PLGA 30-60 KDa, P2191-5G, Sigma, 50:50 (Lactide:Glycolide),
PLA 10 KDa, 764620-5G, Sigma
PLA 30 KDa, 767344-5G, Sigma
PCL 14 KDa, 440752-5G, Sigma,
rat tail tendons, Gibco A1048301,
spironolactone, S3378-1G, Sigma,
dexamethasone, D4902-1G, Sigma, and
aldosterone, A9477-25MG, Sigma.

Unless otherwise indicated, all the materials were used as received from the manufacturers.

The prepared or commercial materials were characterized by transmission electron microscopy (TEM), hydroxyproline assay, measurement of contact angle, measurement of elongation at break, measurement of breaking strength, measurement of elasticity, measurement of toxicity, measurement for monitoring the release of the active ingredient by UV spectroscopy and measurement of the biological activity of the spironolactone.

The analysis by transmission electron microscopy (TEM) was carried out using an apparatus sold under the trade name Technai Spirit G2, by FEI.

The contact angle was measured using a tensiometer sold under the trade name DSA 30 by KRUSS. This measurement was carried out according to the following Wilhelmy method: a solution of organic polymer with a concentration of approximately 160 mg/ml dissolved in tetrahydrofuran (THF) is deposited on a clean and dry glass slide. Drying is carried out in order to form a film of the organic polymer at the surface of the glass slide. A drop of water is then deposited on the glass slide covered with organic polymer. This operation is carried out at approximately 25° C. The tensiometer measures the surface tension between the water and the glass slide covered with organic polymer and calculates the resulting contact angle with the water. The larger the contact angle, the more the organic polymer is hydrophobic.

The elasticity was determined at 25° C. using an apparatus sold under the trade name Electroforce 3220 by Bose.

The shear modulus was determined at 25° C. using an apparatus sold under the trade name MCR 301 by Anton Paar.

The elongation at break was determined at 25° C. using an apparatus sold under the trade name Electroforce 3220 by Bose.

The breaking strength was determined at 25° C. using an apparatus sold under the trade name Electroforce 3220 by Bose.

The hydroxyproline assay makes it possible to determine the concentration of collagen in the different solutions or materials. Hydroxyproline is an amino acid that is heavily present in the collagen polypeptide chain. The assay was carried out in the following manner: the collagen solution to be tested was hydrolysed under acid conditions (HCl 6 M) at approximately 108° C. The hydroxyproline was thus released, then the resulting mixture was dried. The hydroxyproline was oxidized by Chloramine T then complexed with dimethylamino-4-benzaldehyde (DMBA) to give a coloured product. The concentration was determined by spectrophotometric measurement at 557 nm by comparison with a calibration range.

The toxicity of the materials was measured as follows: primary human dermal fibroblasts were seeded onto a culture plate. The composite hydrogels were then incubated with the fibroblasts for 1 or 6 days. The cell viability was measured by a metabolic test (AlamarBlue Assay®) and compared with that of control fibroblasts (without addition of composite hydrogel).

The monitoring of the release of the active ingredient was measured by UV spectroscopy using an apparatus sold under the trade name Uvikon XL, by NorthStar Scientific.

The biological activity of the spironolactone was measured in the following manner: H9C2 cells are myoblasts genetically modified by the incorporation of the reporter gene encoding luciferase downstream of the promoter having sequences binding the complex of aldosterone plus its receptor. When aldosterone was added, luciferase was expressed and detected by bioluminescence. When the spironolactone released by the tested hydrogels is active, its incubation with H9C2s inhibits aldosterone and makes the bioluminescence signal zero. This inhibition reports the biological activity of the spironolactone on H9C2s incubated with aldosterone. The spironolactone activity was measured directly on the spironolactone releasing liquid originating from the different types of hydrogels tested.

### Example 1: Preparation of a Synthetic Composite Biomaterial in Accordance with the First Subject of the Invention

1.1 Extraction of the Collagen

The collagen was extracted from rat tail tendons, known to be very rich in type I collagen. For this purpose, rat tendons were rinsed with phosphate-buffered saline (PBS), centrifuging them for approximately 5 min at approximately 4° C. and at 3000 G (G is the unit of measurement of the centrifugation speed and corresponds to acceleration due to gravity) until the solution becomes clear, devoid of cells and of blood. They were then rinsed with an approximately 4 M solution of NaCl in order to destroy all the remaining cells. After another rinse in PBS to eliminate all traces of NaCl, the washed tendons were mixed with a solution of approximately 0.5 M acetic acid for approximately 24 h, then the resultant mixture was centrifuged at approximately 3000 G for approximately 20 min. The resulting mixture then comprised triple helices of collagen I in the presence of other proteins. The collagen I was selectively precipitated by dropwise addition of an approximately 4 M solution of NaCl to the resulting solution to obtain a final NaCl concentration of 0.7 M. The resulting mixture was then centrifuged at 3000 G and the precipitate obtained was dissolved in 0.5 M acetic acid to form a solution comprising essentially collagen I. This solution was dialysed against the same solvent in order to completely eliminate the NaCl (4 baths of approximately 24 h) and centrifuged at approximately 21 000 G for approximately 3 hours to eliminate the final colloidal aggregates.

The collagen solution prepared in this way was stored at approximately 4° C. in order to conserve its triple helix structure. The assay of hydroxyproline in this solution made it possible to determine its concentration of collagen, which was approximately 5 mg/ml.

1.2 Preparation of a Concentrated Collagen I Hydrogel

The solution of collagen at approximately 5 mg/ml as prepared above was dissolved in an approximately 0.5 M acetic acid solution. The solution was evaporated in the air under a sterile fume hood until a final collagen concentration of approximately 40 mg/ml was achieved. The evaporation of the solvent was monitored by weighing. The final concentration of the solution was then confirmed by hydroxyproline assay.

The solution of collagen at approximately 40 mg/ml obtained in this way was introduced into a mould, then the mould/collagen solution assembly was centrifuged in order to flatten out irregularities. The resulting mould was then placed under ammonia vapours for approximately 12 h, to enable gelling and fibrillogenesis of the collagen I. The hydrogel obtained was rinsed several times in sterile PBS baths (i.e. which had undergone moist sterilization in an autoclave) to eliminate the ammonia and bring the pH to 7. The pH was monitored before each washing. The pure collagen hydrogel obtained is denoted M_{A-H}.

1.3 Dehydration of a Concentrated Collagen I Hydrogel

The collagen I hydrogel as obtained above was dehydrated gradually by incubation in several successive mixed THF/water baths: a THF/water bath having a concentration by volume of THF of 30%, a THF/water bath having a concentration by volume of THF of 50%, a THF/water bath having a concentration by volume of THF of 70%, a THF/water bath having a concentration by volume of THF of 95% and finally a pure THF bath. Incubation in each of the baths lasted approximately 1 h.

Following the dehydration step, a dehydrated pure collagen hydrogel M_{A-HD} was obtained.

The dehydrated pure collagen hydrogel M_{A-HD} can be rinsed with PBS and lyophilized to give a dry pure collagen material M_{A-S} then M_{A-S} can be rehydrated with PBS to re-form a pure collagen hydrogel denoted M_{A-HR}.

1.4 Preparation of the Synthetic Composite Biomaterial M₁

A solution comprising 160 mg/ml of PLGA 30-60 kDa and 10⁻² M (i.e. 4.16 mg/ml) of spironolactone was prepared by dissolving the spironolactone in THF then by dissolving the PLGA in the preceding mixture.

The dehydrated pure collagen hydrogel M_{A-HD} as prepared above was impregnated for approximately 12 h with the solution comprising the PLGA and the spironolactone, the volume of the solution being at least 5 times greater than that of the dehydrated pure collagen hydrogel.

After impregnation, the composite hydrogel obtained was rinsed 3 times for approximately 30 seconds with pure THF to eliminate the excess PLGA, then 3 times for approximately 30 minutes with sterile PBS to fix the polymer within the fibrillar collagen network and form the composite biomaterial of the invention M_{1-H} in the form of a composite hydrogel.

A dry form of said composite biomaterial was also obtained. For this purpose, the composite biomaterial M_{1-H} was submerged in liquid nitrogen for approximately 10 min in order to avoid the formation of ice crystals, then lyophilized for approximately 24 h (temperature of less than −40° C., vacuum at 100 μBarr), to form the composite biomaterial of the invention M₁₋₅ in the form of a dry composite material.

The dry composite material was then rehydrated by addition of a phosphate-buffered saline to form a material M_{1-HR} in the form of a composite hydrogel.

The collagen/organic polymer weight ratio in said biocomposite material was 1/1.

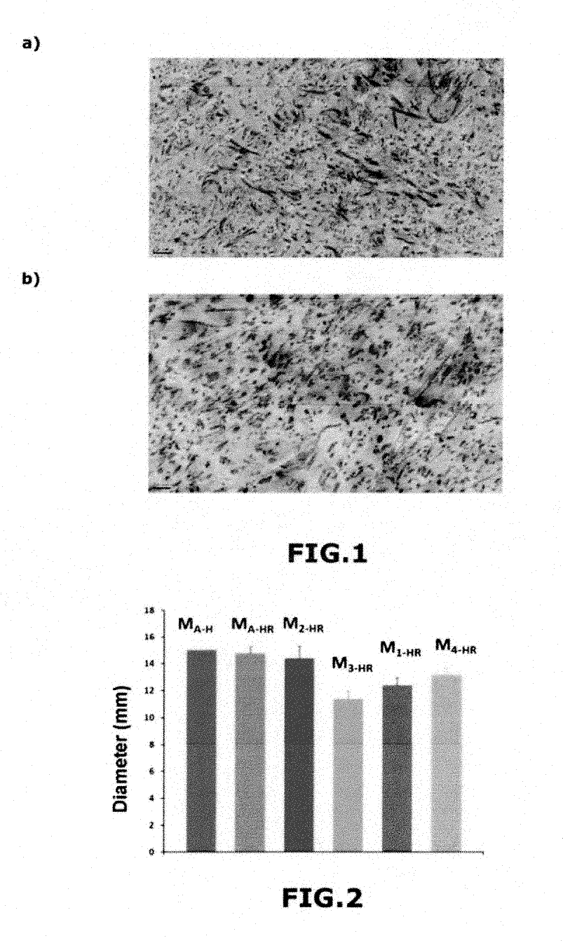

FIG. 1 shows a transmission electron micrograph of a pure collagen hydrogel M_{A-H} as obtained in example 1.2 (FIG. 1a) which does not form part of the invention, and a micrograph of the composite biomaterial M_{1-HR} in the form of a composite hydrogel in accordance with the invention, obtained in example 1.4 (FIG. 1b).

FIG. 1 shows a homogeneous composite biomaterial in which the fibrillar and striated structure of the collagen has been preserved. Moreover, microscopic domains of PLGA polymer cannot be discerned, which means that the polymer is uniformly distributed within the collagen.

The table below summarizes the values of the contact angles of the different organic polymers used in example 1 and the examples below:

| | PLGA | PLGA | PLGA | PCL |
|---|---|---|---|---|
| | 7-17 kDa | 24-38 kDa | 30-60 kDa | 14 kDa |
| Contact angle | 68.73 | 70.03 | 71.03 | 109.33 |
| (°) | | | | |
| Sum of the mean | 0.78 | 0.49 | 0.51 | 1.49 |
| deviations (°) | | | | |

### Example 2: Preparation of Other Synthetic Composite Biomaterials in Accordance with the First Subject of the Invention

Composite biomaterials M_{2-HR}, M_{3-HR} and M_{4-HR} were prepared by impregnating the dehydrated pure collagen hydrogel M_{A}-no as obtained in example 1.3 above with the following respective impregnation solutions:
a solution comprising approximately 10⁻² M of spironolactone and approximately 160 mg/ml of PLGA 7-17 kDa,
a solution comprising approximately 10⁻² M of spironolactone and approximately 160 mg/ml of PLGA 24-38 kDa, and
a solution comprising approximately 10⁻² M of spironolactone and approximately 160 mg/ml of PCL 14 kDa.

The steps of impregnation, rinsing, lyophilization and rehydration are identical to those described in example 1.4 above.

FIG. 2 represents the diameter (in mm) of the composite biomaterials in the form of composite hydrogels in accordance with the invention M_{1-HR}, M_{2-HR}, M_{3-HR} and M_{4-HR} and, by way of comparison, the diameter of a pure collagen hydrogel not in accordance with the invention M_{A-H} as obtained in example 1.2 and of a pure collagen hydrogel M_{A-HR} as obtained in example 1.3.

FIG. 2 shows that the presence in the hydrogel of an active ingredient and a hydrophobic organic polymer as defined in the invention has only very little influence on the diameter of the hydrogel obtained, or no influence at all. The composite hydrogels therefore have a good capacity to be hydrated, close to that of pure collagen, and do not retract.

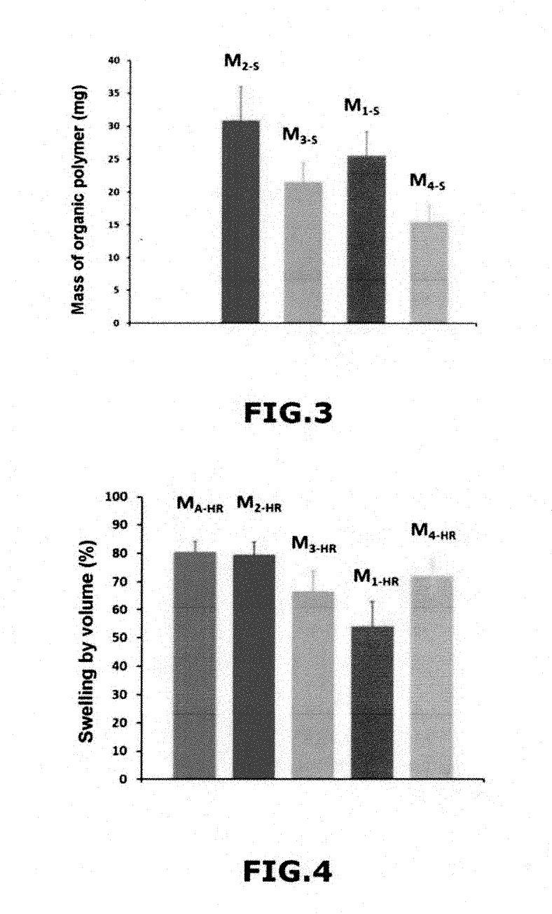

FIG. 3 represents the mass of organic polymer (in mg) present in the composite biomaterials in the form of dry composite materials in accordance with the invention M_{1-S}, M_{2-S}, M_{3-S} and M_{4-S}.

FIG. 3 shows that the composite biomaterial of the invention may incorporate a greater amount of organic polymer when the PLGA 7-17 kDa is used (M_{2-S}). The capacities of integration of the organic polymers tested are nonetheless suitable and are greater than approximately 15 mg, and preferably range approximately from 20 to 31 mg for a mass of collagen of the order of 25 mg.

FIG. 4 represents the swelling by volume of the composite biomaterials in the form of composite hydrogels in accordance with the invention M_{1-HR}, M_{2-HR}, M_{3-HR} and M_{4-HR} and, by way of comparison, of a pure collagen hydrogel M_{A-HR} as obtained in example 1.3 (as % relative to the initial volume of the pure collagen hydrogel M_{A-H}).

FIG. 4 shows a good capacity for swelling of the composite hydrogels in accordance with the invention (approximately 55% to 80%), even similar to that of a pure collagen hydrogel when PLGA 7-17 kDa is used.

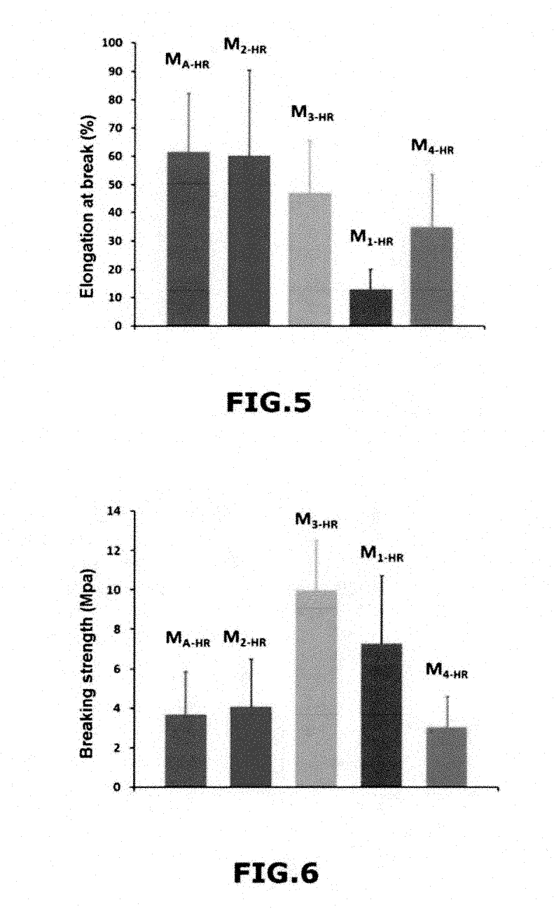

FIG. 5 represents the elongation at break of the composite biomaterials in the form of composite hydrogels in accordance with the invention M_{1-HR}, M_{2-HR}, M_{3-HR} and M_{4-HR} and, by way of comparison, that of a pure collagen hydrogel M_{A-HR} as obtained in example 1.3 (as % relative to the initial length of each of the materials tested).

FIG. 6 represents the breaking strength (in MPa) of the composite biomaterials in the form of composite hydrogels in accordance with the invention M_{1-HR}, M_{2-HR}, M_{3-HR} and M_{4-HR} and, by way of comparison, that of a pure collagen hydrogel M_{A-HR} as obtained in example 1.3.

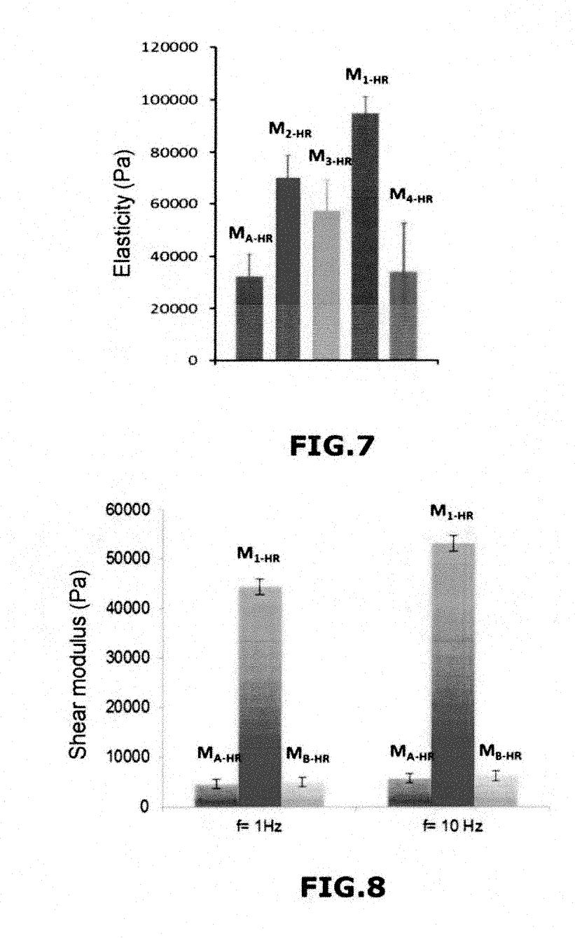

FIG. 7 represents the elasticity (in Pa) of the composite biomaterials (Young's modulus) in the form of composite hydrogels in accordance with the invention M_{1-HR}, M_{2-HR}, M_{3-HR} and M_{4-HR} and, by way of comparison, that of a pure collagen hydrogel M_{A-HR} as obtained in example 1.3.

FIG. 8 represents the shear modulus (in Pa) at different frequencies (approximately 1 Hz and 10 Hz) of the composite biomaterial in the form of a composite hydrogel in accordance with the invention M_{1-HR}, and, by way of comparison, that of a pure collagen hydrogel M_{A-HR} as obtained in example 1.3 and that of a collagen hydrogel impregnated by spironolactone (i.e. without organic polymer) M_{B-HR} obtained from the dehydrated pure collagen material M_{A-HD} as obtained in example 1.3 which has been impregnated with a solution comprising approximately 10⁻² M of spironolactone in THF and rinsed, lyophilized and rehydrated in accordance with example 1.4 above. Thus, a significant improvement in the mechanical strength of the composite biomaterial of the invention is observed compared to a pure collagen hydrogel or a pure collagen hydrogel comprising spironolactone.

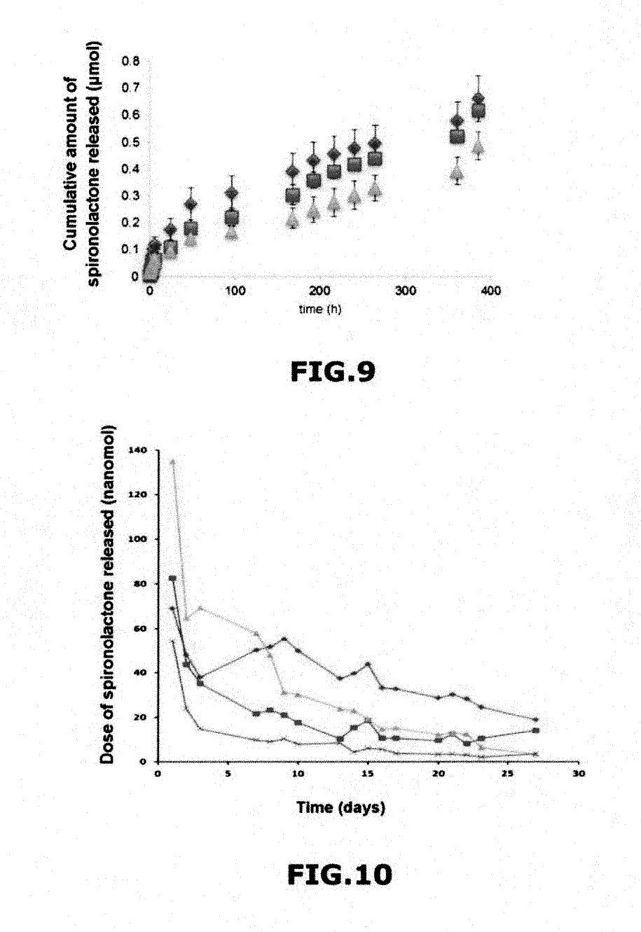

FIG. 9 represents the profile of release of spironolactone of the composite biomaterials in the form of dry composite materials in accordance with the invention M_{1-S} (curve with triangles), M_{1-S1} (curve with diamonds) and M_{1-S2} (curve with squares). The different curves show the cumulative amount of spironolactone released (in nmol) as a function of time (in hours).

It is worth noting that the biomaterials initially used in this experiment are in the form of dry composite materials. However, they are instantly rehydrated during the measurement for monitoring the release of the active ingredient. The same applies for the measurements of the toxicity of the biomaterials and the biological activity of spironolactone, as described below.

The composite biomaterials M_{1-S1} and M_{1-S2} were prepared as in example 1 except in terms of the concentration of PLGA in the impregnation solution which was approximately 40 mg/ml for M_{1-S1} and approximately 80 mg/ml for M_{1-S2} (Instead of approximately 160 mg/ml for M_{1-S}).

It is observed that a concentration of approximately 160 mg/ml is preferred for promoting the release of a constant dose of spironolactone over time and for obtaining better control of the release of spironolactone. It is worth noting that the three composite biomaterials in accordance with the invention only released approximately from 30 to 60% of spironolactone in 400 h (approximately 2 weeks).

FIG. 10 represents the profile of release of spironolactone of the composite biomaterials of the invention M_{1-S} (curve with crosses), M_{1-S} (curve with diamonds), M_{3-S} (curve with squares) and M_{4-S} (curve with triangles). The different curves show the daily dose of spironolactone released (as nanomol per day) as a function of time (in days).

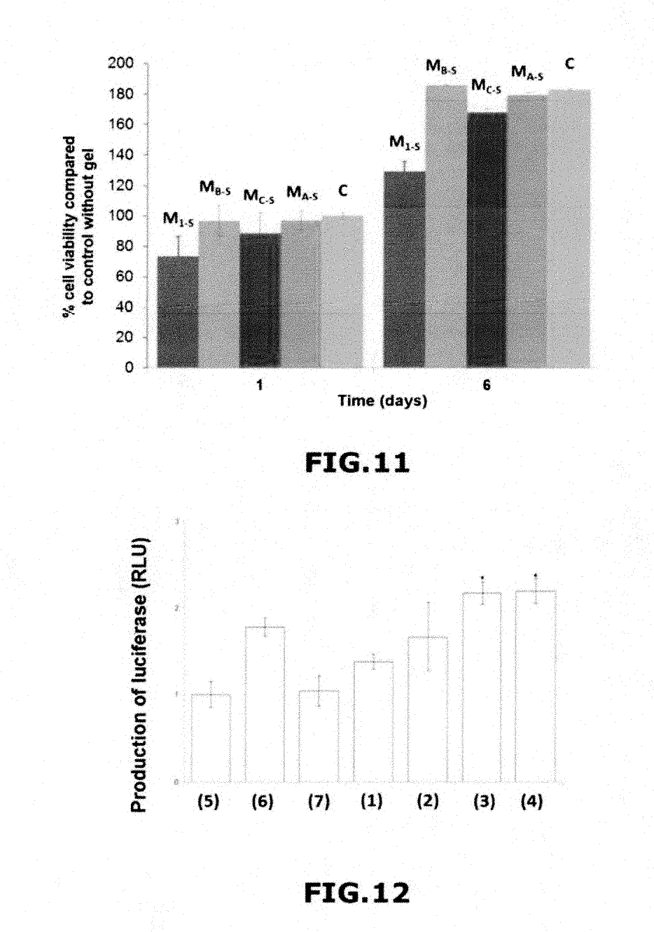

FIG. 11 represents the toxicity of the composite biomaterial of the invention M_{1-S} on cells (fibroblasts) and by way of comparison:
the toxicity of a dry collagen material impregnated with spironolactone M_{B-S} obtained from the dehydrated pure collagen material M_{A-HD} as obtained in example 1.3 which was impregnated with a solution comprising approximately 10⁻² M of spironolactone in THF, rinsed and lyophilized in accordance with example 1.4 above,
the toxicity of a dry collagen material impregnated with PLGA M_{C-S} obtained from the dehydrated pure collagen material M_{A-HD} as obtained in example 1.3 which was impregnated with a solution comprising approximately 160 mg/ml of PLGA 30-60 kDa in THF, rinsed and lyophilized in accordance with example 1.4, and
the toxicity of a dry pure collagen material M_{A-S} as obtained in example 1.3.

Moreover, C represents a negative control (well containing just the cells without hydrogel). The different charts show the cell viability of the materials M_{1-S}, M_{B-S}, M_{C-S} and M_{A-S} as defined above (in %) at 1 day and at 6 days, relative to the negative control C.

On the sixth day, all the points are above 100% since the cells have proliferated and the study is cumulative. It is observed that the spironolactone has a slight toxic effect on the cells since the two triangles are slightly below the control (circles). Indeed, on the first day, only 74% of the cells survived. However, this does not prevent cell proliferation (the straight lines are increasing). FIG. 11 also shows the non-toxicity associated with the hydrophobic organic polymer PLGA since the squares are at the same level as the control (circles).

FIG. 12 represents the biological activity of the spironolactone released from a hydrogel after 15 days of incubation in PBS (with changing of the buffer every day); when the following are used:

(1) a mixture of a composite biomaterial in accordance with the invention M_{1-S} with aldosterone at approximately 10⁻⁸ M, and by way of comparison:

(2) a mixture of a material M_{B-S} with aldosterone at approximately 10⁻⁸ M,

(3) a mixture of a material M_{C-S} with aldosterone at approximately 10⁻⁸ M,

(4) a mixture of a material M_{A-S} with aldosterone at approximately 10⁻⁸ M,

(5) a negative control (well containing the culture medium without aldosterone),

(6) aldosterone at approximately 10⁻⁸ M, and

(7) a mixture of aldosterone at approximately 10⁻⁸ M and spironolactone at approximately 10⁻⁶ M.

FIG. 12 shows the production of luciferase (in relative light units or RLU) depending on the type of medium used. It is observed that the luciferase activity is greater under the conditions (6) than under the conditions (5), meaning that the aldosterone has indeed bound to the mineralocorticoid receptor and caused strong transcription. When spironolactone at approximately 10⁻⁶ M (conditions (7)) is added, the effect of the aldosterone is inhibited and the receptor is not activated. The conditions (1) using the composite biomaterial M_{1-S} in accordance with the invention show comparable activity to that obtained under the conditions (7), indicating that spironolactone has indeed been released by the composite biomaterial M_{1-S}, while retaining the activity thereof. When there is no PLGA (conditions (2)), the collagen does not retain the spironolactone and the remaining amount of spironolactone is inactive or of relatively low concentration to act against aldosterone. Finally, the two final conditions (3) and (4) correspond to controls without spironolactone and are at the same height as that associated with the conditions (6) containing solely aldosterone at 10⁻⁸ M; there is therefore no effect of the collagen hydrogel, nor of the collagen hydrogel comprising PLGA.

### Example 3: Preparation of Other Synthetic Composite Biomaterials in Accordance with the First Subject of the Invention

Composite biomaterials M_{5-HR} and M_{6-HR} were prepared by impregnating the dehydrated pure collagen hydrogel M_{A-HD} as obtained in example 1.3 above with the following respective impregnation solutions:
a solution comprising approximately 10⁻² M of spironolactone and approximately 160 mg/ml of PLA 10 kDa, and
a solution comprising approximately 10⁻² M of spironolactone and approximately 160 mg/ml of PLA 30 kDa.

The steps of impregnation, rinsing, lyophilization and rehydration are identical to those described in example 1.4 above.

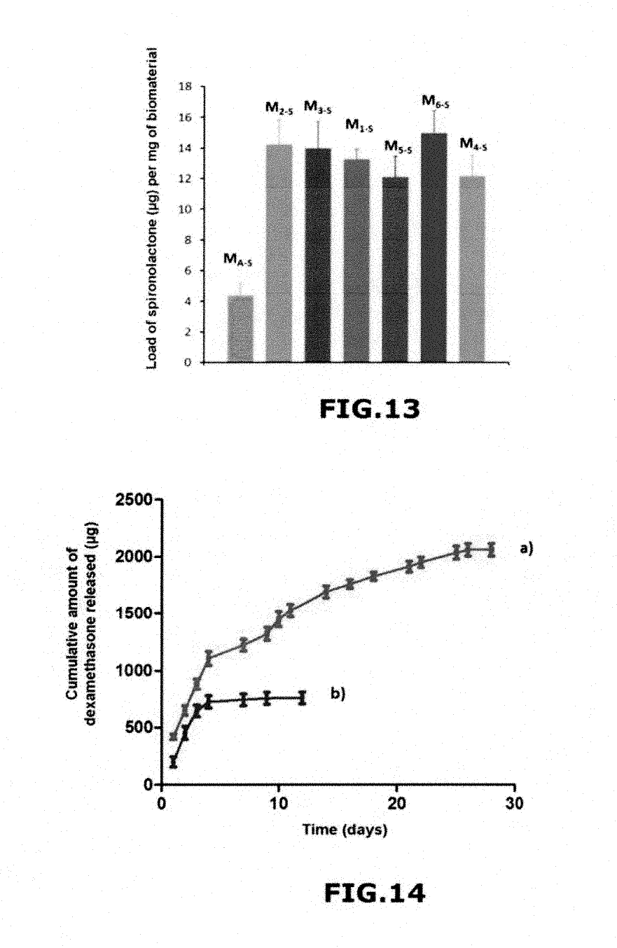

FIG. 13 shows the load of spironolactone (in μg) of the composite biomaterials in the form of dry composite materials in accordance with the invention M_{1-S}, M_{2-S}, M_{3-S}, M_{4-S}, M_{5-S} and M_{6-S}, per mg of composite biomaterial, and by way of comparison the load of spironolactone (in μg) of the dry pure collagen material M_{A-S} per mg of said material.

Other composite biomaterials M_{7-HR} and M_{8-HR} were prepared by impregnating the dehydrated pure collagen hydrogel M_{A-HD} as obtained in example 1.3 above with the following respective impregnation solutions:
a solution comprising approximately 5.3×10⁻² M (i.e. 21 mg/ml) of dexamethasone and approximately 160 mg/ml of PLGA 7-17 kDa, and
a solution comprising approximately 3.2×10⁻² M (i.e. 12.6 mg/ml) of dexamethasone and approximately 160 mg/ml of PLGA 7-17 kDa.

The steps of impregnation, rinsing, lyophilization and rehydration are identical to those described in example 1.4 above.

FIG. 14 shows the profile of release of dexamethasone of the composite biomaterials in the form of dry composite materials in accordance with the invention M_{7-S} (FIG. 14a)) and M_{8-S} (FIG. 14b)). The different curves show the cumulative amount of dexamethasone released (in μg) as a function of time (in days).

1. Synthetic composite biomaterial comprising:
collagen, at least one organic polymer and at least one active ingredient, wherein:
the organic polymer is biodegradable, biocompatible, hydrophobic and has a glass transition temperature of less than or equal to 50° C. and a mean molar mass ranging from 5 to 120 kDa,
the collagen is in the form of striated fibrils, in which the periodicity of the striations is 67 nm,
the collagen/organic polymer weight ratio ranges from 10/1 to 1/3,
the active ingredient is a hydrophobic active ingredient chosen from anti-inflammatories, antibiotics, compounds promoting tissue repair or wound healing, and a mixture thereof.

2. Biomaterial according to claim 1, wherein said biomaterial is in the form of a composite hydrogel and comprises from 70 to 95% by weight of water relative to the total weight of the composite biomaterial.

3. Biomaterial according to claim 2, wherein said biomaterial has an elasticity ranging from 50 000 Pa to 100 000 Pa.

4. Biomaterial according to claim 2, wherein said biomaterial has an elongation at break ranging from 40 to 75%.

5. Biomaterial according to claim 1, wherein said biomaterial is in the form of a dry composite material and comprises at most 10% by weight of water relative to the total weight of the composite biomaterial.

6. Biomaterial according to claim 1, wherein the organic polymer is chosen from aliphatic polyesters, polyethylene glycols, polyanhydrides and poly(ortho-esters).

7. Biomaterial according to claim 1, wherein the organic polymer is an aliphatic polyester chosen from a polyglycolide, a polylactide, a copolymer of glycolide and lactide, a polylactone and a polyhydroxyalkanoate.

8. Biomaterial according to claim 1, wherein the organic polymer is in the form of nanodomains having a mean size of less than or equal to 700 nm.

9. Process for preparing a composite biomaterial as defined in claim 1, comprising at least the following steps:
i) preparing a hydrogel of collagen in the form of striated fibrils in which the periodicity of the striations is 67 nm, the concentration of collagen in the hydrogel being at least 10 mg/ml,
ii) dehydrating the hydrogel as prepared in step i) by incubating said hydrogel in several successive mixed solutions having an increasing proportion of organic solvent, said aqueous and organic solvents being miscible, followed by a final incubation in a pure solution of said organic solvent,
iii) bringing the dehydrated hydrogel from step ii) into contact with an impregnation solution comprising at least one organic polymer and at least one active ingredient as defined in any one of claims 1 to 8, the volume of impregnation solution/volume of dehydrated hydrogel volume ratio being greater than or equal to 3,
iv) rinsing the impregnated hydrogel from step iii) with an organic solvent, then with an aqueous solvent.

10. Process according to claim 9, wherein step i) is carried out according to the following sub-steps:
i-1) preparing a solution of acid-soluble collagen, the collagen content of which varies from 1 to 5 mg/ml,
i-2) evaporating the solution from step i-1) in air, and
i-3) bringing the solution from step i-2) into contact with a base.

11. Process according to claim 9, wherein step ii) is carried out by incubating the hydrogel from step i) in a mixed organic solvent/aqueous solvent solution in which the content of organic solvent is from 20 to 30% by volume, then in a mixed organic solvent/aqueous solvent solution in which the content of organic solvent is from 40 to 50% by volume, then in a mixed organic solvent/aqueous solvent solution in which the content of organic solvent is from 60 to 70% by volume, then in a mixed organic solvent/aqueous solvent solution in which the content of organic solvent is from 80 to 95% by volume, then in a pure solution of said organic solvent.

12. Process according to claim 9, wherein the incubation of step ii) in each of the solutions lasts from 30 min to 2 h.

13. Process according to claim 9, wherein the concentration of organic polymer of the impregnation solution ranges from 20 to 500 mg/ml.

14. Process according to claim 9, wherein said process also comprises a step v) of lyophilization of the composite biomaterial of step iv).

15. Composite biomaterial as defined in claim 1, for medical use thereof.

16. Composite biomaterial as defined in claim 1, for use thereof in the treatment of chronic wounds.

17. Composite biomaterial as defined in claim 1, for use thereof in the preventative treatment of infections after cardiac or colorectal surgery.

18. Composite biomaterial as defined in claim 1, for use thereof in the treatment of hernias of the abdominal wall or eventrations.

19. Therapeutic dressing comprising:
an internal layer and an external layer of a secondary dressing chosen from an adhesive, a compress, a bandage and a mixture thereof, wherein the internal layer comprises a composite biomaterial as defined in claim 1.

20. Abdominal wall reinforcement, wherein said abdominal wall reinforcement comprises a composite biomaterial as defined in claim 1.
